(19) United States
(12) Patent Application Publication
(10) Pub. No.: US 2015/0140528 A1
(43) Pub. Date: May 21, 2015

(54) METHOD, ARRANGEMENT AND COMPUTER PROGRAM FOR ENHANCING COGNITIVE FUNCTION OF A USER

(71) Applicants: Sverker Sikström, Lund (SE); Göran Söderlund, Sogndal (NO)

(72) Inventors: Sverker Sikström, Lund (SE); Göran Söderlund, Sogndal (NO)

(21) Appl. No.: 14/386,745

(22) PCT Filed: Jul. 23, 2012
(86) PCT No.: PCT/SE2012/000114

§ 371 Date: Jan. 9, 2015

(30) Foreign Application Priority Data
Jul. 22, 2011 (SE) 1150718-3

(51) Int. Cl. G09B 19/00; A61B 5/16

(52) U.S. Cl. CPC G09B 19/00; A61B 5/168
USPC 434/236

(57) ABSTRACT

Method, arrangement and computer program in a noise generator (100), for enhancing a cognitive function of a user (110). The method comprises estimating (201) attention level of the user (110), determining (202) an optimal cognitive performance level of the user (110), predicting (203) a noise amount and amplitude that is associated with the optimal cognitive performance level of the user (110), where this prediction is based on the estimated (201) attention level of the user (110), and emitting (204) noise having the predicted (203) noise amount and amplitude, to be received by the user (110).

### TECHNICAL FIELD

The present disclosure relates to a method, an arrangement and a computer program. More in particular, it relates to a mechanism for enhancing a cognitive function of a user.

### BACKGROUND

In order to perceive and understand the world around us, we somehow have to structure and filter the stimuli we constantly are flooded by, as our human brain is only capable of handling a limited amount of incoming stimuli simultaneously.

Thereby, attention is given to some particular events that we consider important, such as for example a sudden glimpse in the corner of the eye of a rapidly approaching football, while we routinely filter out every-day events that have no impact or relevance for our doings. The cognitive process of concentrating on one aspect of our environment while ignoring other aspects may be referred to as attention.

An applied example of attention is when we are conversing with someone at a social event. Normally we can focus on listening to our conversing partner and filtering out other irrelevant surrounding conversations. This effect is sometimes referred to as the cocktail party effect.

The above-described cognitive mechanism is a skill that humans have without any particular training.

However, some people suffer from attention defects. This may be observed in children or adolescents, as well as adults, who for example have problems attending instructions or assignments given in a school environment. People suffering from such attention deficit may be perceived as careless and unattended by the surrounding; they seem not to be listening when talked to, frequently fail to finish given assignments, appear absentminded and forgetful and they may easily be distracted from finishing their doings.

In fact, lack of ability to pay attention is a most limiting handicap for the people suffering from such attention deficit, as it hinders them from education and studies. This may occur also when the individual does not suffer from any decreased intellectual capacity per se. It may also be a social handicap preventing normal socialization and human interaction.

It has even been suggested that the inability of some children to resolve conflicts is generally because they lack executive control of their attention.

It is thus a problem to help people suffering from attention disorder to attend and focus on the task that they desire to attend upon.

An example of a somewhat related problem that may be experienced to any human, may appear for example in an open-plan office where it may be difficult to focus on, and fully attend to the reading of a document while annoying speech sounds irregularly intrude the attention of the human.

Open-plan offices frequently have a tendency be either too quiet, where someone dropping a pen in the next cubicle is perceived as most distracting; or being too noisy, where the conjunct cacophony from intermittent conversations of others in the office, ringing phones and pumping printers render it impossible to concentrate.

Yet another problem that also may be experienced to any human, also if having a normal attention capability is to perceive subtle cognitive stimuli, which cognitive stimuli lays below our threshold for perceiving cognitive stimuli. Any such threshold for perceiving cognitive stimuli is individual, and may to some degree be age related and may further be different for different kinds of cognitive stimuli. For example, a sound may be too faint to be detected by humans, e.g., the sound of a game animal moving away undetected from a human hunter.

Further, to mention another example comprising visual cognition, a person driving a vehicle such as a car at night time may have problems to detect an obstacle on the road, such as a crossing animal, in time to stop the vehicle before impact as a result of lack of attention.

To summarize, in many situations there is a problem for humans to pay appropriate attention, which may have devastating results, both for the individual as well as for the surroundings.

### SUMMARY

It is the object to obviate at least some of the above disadvantages and provide an improved mechanism for enhancing cognitive function of a user.

According to a first aspect, the object is achieved by a method using a noise generator. The method aims at enhancing cognitive functions of a user. The method comprises estimating attention level of the user. Also, the method comprises determining an optimal cognitive performance level of the user in absence of noise. Further, the method comprises predicting a noise amplitude and an amount of noise that is associated with the optimal cognitive performance level of the user, where this prediction is based on the estimated attention level of the user. Also, the method comprises emitting the predicted amount of noise having the predicted noise amplitude, to be received by the user.

According to a second aspect, the object is also achieved by an arrangement in a noise generator. The arrangement is configured to perform the method for enhancing a cognitive function of a user. The arrangement comprises a processing circuit configured to estimate an attention level of the user. Also, the processing circuit is configured to determine an optimal cognitive performance level of the user in absence of noise. Further, the processing circuit is configured to predict an amount of noise and a noise amplitude that is associated with the optimal cognitive performance level of the user, based on the estimated attention level of the user. Further, the arrangement comprises a noise-emitting unit, configured to emit the predicted amount of noise having the predicted noise amplitude, to be received by the user.

According to a third aspect, the object is also achieved by a computer program for enhancing a cognitive function of a user. The computer program is configured to estimate the attention level of the user. Also, the computer program is configured to determine the optimal cognitive performance level of the user in absence of noise. Further, the computer program is configured to predict a noise amplitude and an amount of noise that is associated with the optimal cognitive performance level of the user, where this prediction is based on the estimated attention level of the user. Also, the computer program is configured to emit the predicted amount of noise having the predicted noise amplitude, to be received by the user, when being loaded into a processing circuit.

Thanks to embodiments of the herein disclosed methods, arrangements and computer programs, a mechanism is provided, for enhancing cognitive functions of a user, by applying an appropriate amount of noise in a controlled manner. Thereby is the cognitive ability of the user enhanced.

Other objects, advantages and novel features of the methods, arrangements and computer programs will become apparent from the following detailed description.

### BRIEF DESCRIPTION OF THE DRAWINGS

The methods, arrangements and computer program will subsequently be described in more detail in relation to the enclosed drawings, in which:

FIG. 1 is a block diagram illustrating an embodiment of a noise generator.

FIG. 2 is a flow chart illustrating an embodiment of the method.

FIG. 3A is a block diagram illustrating an embodiment of a noise generator.

FIG. 3B is a block diagram illustrating an embodiment of a noise generator.

FIG. 3C is a block diagram illustrating embodiments of an arrangement in a noise generator.

### DETAILED DESCRIPTION

It is herein disclosed a method, a computer program an arrangement and a computer program in a noise generator for enhancing a cognitive function of a user, which may be put into practice in the embodiments described below. Those methods, computer programs and arrangements may, however, be embodied in many different forms and are not to be considered as limited to the embodiments set forth herein; rather, these embodiments are provided so that this disclosure will be thorough and complete.

Still other features and advantages of embodiments of the present methods, computer programs and arrangements may become apparent from the following detailed description considered in conjunction with the accompanying drawings. It is to be understood, however, that the drawings are designed solely for purposes of illustration and not as a definition of the limits of the present methods, computer programs and arrangements. It is further to be understood that the drawings are not necessarily drawn to scale and that, unless otherwise indicated, they are merely intended to conceptually illustrate the structures and procedures described herein.

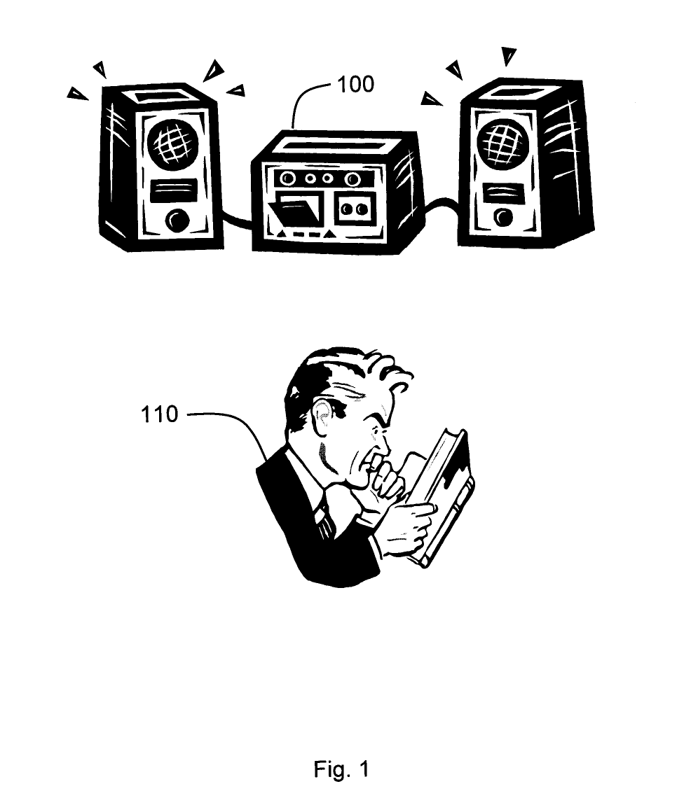

FIG. 1 is a schematic illustration over an event over a period of time, according to some embodiments.

A non-limiting embodiment of a noise generator 100 is illustrated, which is emitting auditory noise to be received by a user 110. The user 110 is focusing on a cognitive task, such as for example studying a book, but it may be any cognitive task. The noise generator 100 may be further referred to as e.g. a cognitive noise generator, a noise machine and/or a white noise generator according to some terminology.

The purpose of the emitted noise is to enhance the cognitive function of the user 110, such as e.g. helping him to focus on the studies, improve memory, enhance the attention, and/or improve the perception of the user 110.

This may be an advantage in particular for example for students, people working in an open office environment, or in fact anyone working or trying to concentrate on an assignment.

However, for some users 110, the added noise serves the purpose of helping the user 110 to perceive stimuli that lay below his/her perception level. The added noise seems to, in conjunction with the subliminal stimuli, add to each other, such that the subliminal stimuli exceed the perception level of the user 110 and thereby becomes recognisable for the user 110.

This may be an advantage in particular for example for anyone working with surveillance, military personnel, guards etc, but also for example vehicle drivers, such as car drivers or pilots.

Thereby, thanks to embodiments of the invention wherein noise is added, a pilot may detect e.g. a suspicious subliminal resonance in an aeroplane, drawing his/hers attention to for example a fault engine part etc., or just in order to increase attention when performance deteriorates.

According to some embodiments, performance deterioration among vehicle drivers may be monitored and measured. Thereby, attentional disruption linked to e.g. sleepiness, or a change in driving behaviour caused for example by the sudden occurrence of events in the driving environment may be determined. Thanks to some embodiments, an appropriate level and amplitude of noise may be emitted for the user 110, thereby modifying (increasing) his/her focus or attention. To mention just one single example, a user may receive a cell phone call while driving. This may be seen as an example of a potentially dangerous situation, as the risk of the driver's attentional disruption increase. When noticing the occurrence of such performance deterioration, or high risk event, a noise may be emitted, sharpening the cognitive performance of the user 110, enabling him/her to focus on the driving environment, for example while e.g. momentarily talking in the cell phone.

It is to be noted that the noise generator 100 is not limited to comprise merely emitting of auditory noise. In different embodiments, the noise generator 100 may be configured to instead, or in simultaneous addition, generate and emit visual noise, tactile noise, vestibular noise, haptic noise and/or subliminal noise. Further, some embodiments of the noise generator 100 may be configured for emitting sound to be received by the user 110 through any of vestibular stimulation to the brain of the user 110, or Transcranial Magnetic Stimulation (TMS).

TMS is a method using electromagnetic induction to induce weak electric currents using a rapidly changing magnetic field for causing depolarisation and/or hyperpolarisation in the neurons of the user's brain.

The noise that is emitted may comprise e.g. white noise, pink noise, brown noise, red noise, flicker noise, random walk noise, Johnson-Nyquist noise, thermal noise, Shot noise, music, sound of rain, just to mention some examples.

The functionality of embodiments of the methods and arrangements herein for emitting noise, such as why it is possible to enhance the cognitive function of the user 110, may to some extent be explained by an analogy. Assume that someone intermittently is toggling a flashlight on and off in a dark chamber. The light shift would be perceived as most distracting to most humans. However, if the light of the chamber is put on, and the flashlight continues to be toggled on and off, it would probably be perceived as less distracting, as the added noise in form of light is covering the distracting flashes from the toggling flashlight and rendering it less intrusive. Thus the added noise according to some embodiments may enhance the user's concentration level.

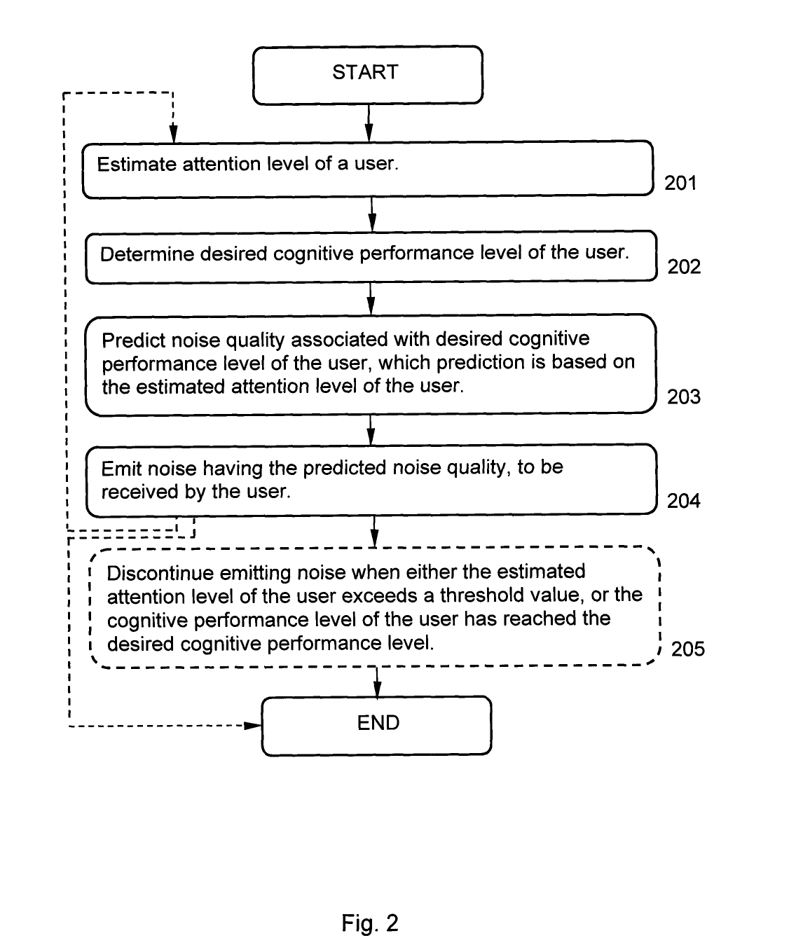

The method according to embodiments of the invention will be further discussed and explained in conjunction with presentation of FIG. 2.

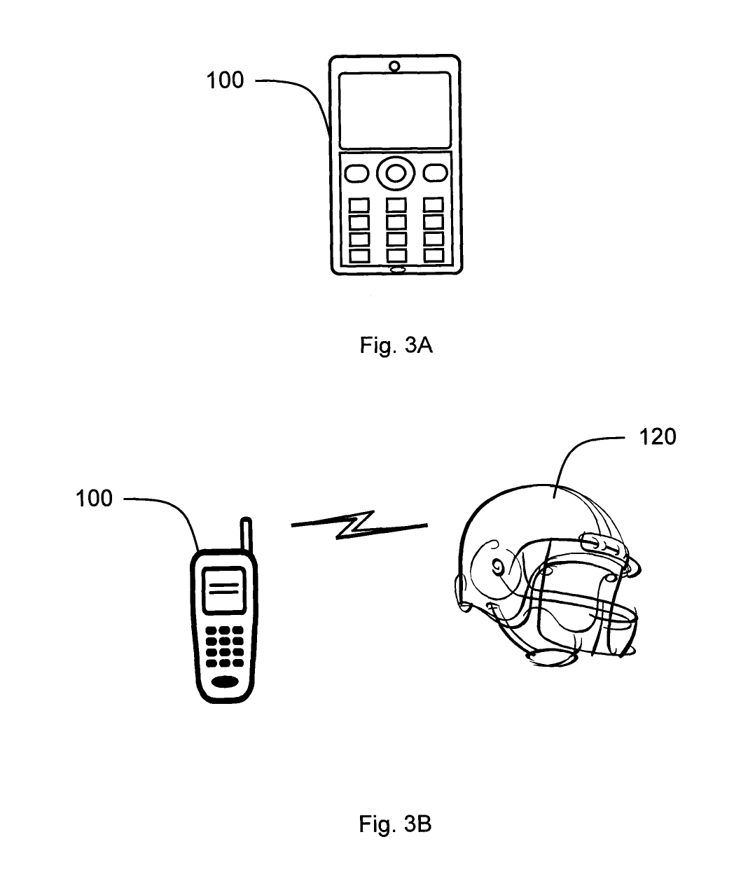

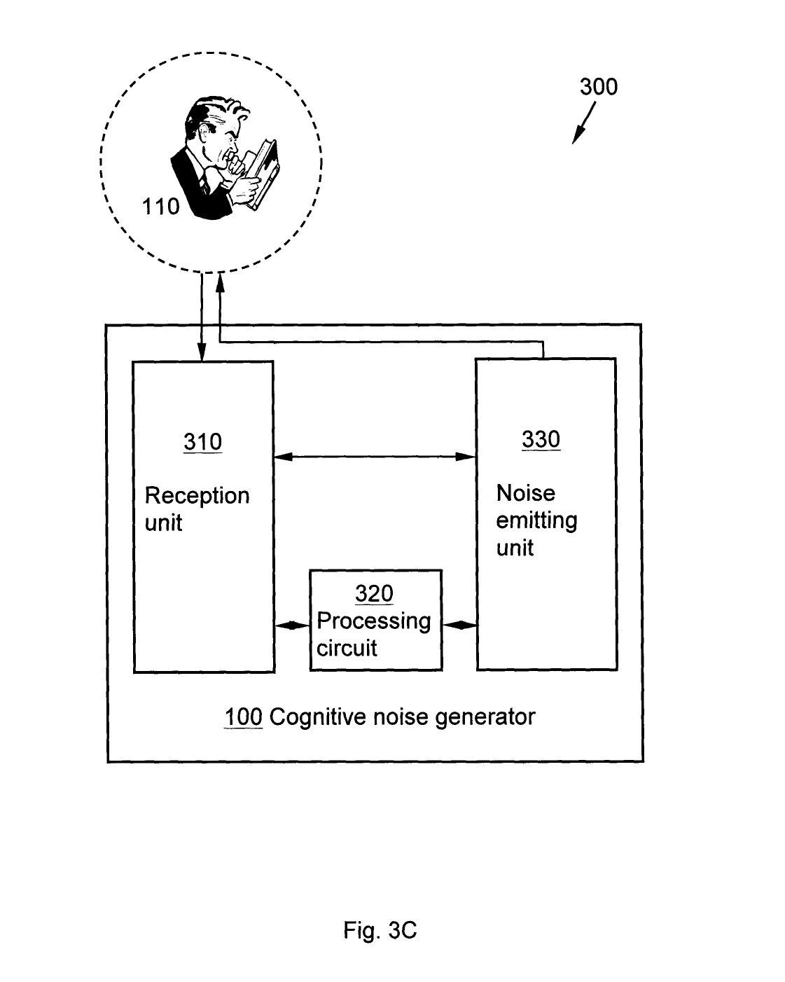

Some different embodiments of the noise generator 100 and an arrangement in the noise generator 100 will be further presented in conjunction with the presentation of FIGS. 3A, 3B and 3C. Also an embodiment of the invention comprising a computer program will be further presented.

However, firstly, a more detailed discussion of what is meant with noise in the present context, and how it may be applied onto the user 110.

When noise is referred to herein, it may comprise perception stimuli emitted in form of e.g. sound, or auditory noise. Such auditory noise may comprise e.g. white noise according to some embodiments. The nature of white noise will be subsequently described more in detail, but it may here be mentioned that the emitted auditory noise may be perceived by the user 110 as a sough or similar according to embodiments. It may be emitted with an intensity of e.g. 70-80 dB, but this is a parameter that is to be set individually, depending on the user 110, as will be further discussed when presenting the method as illustrated in FIG. 2. However, the emitted auditory noise may according to some embodiments comprise e.g. music, sounds of rain, wind and/or waterfall, sounds of ocean waves, animal cry, friction created from a rotating fan, chokings of a tumbler, sound of burning fire, sound of cicada or cricket, bird song, rustle of dry leaves, floor squeak, inhale/exhale sounds from a large mammal, the contact sound of a cetacean, heartbeat of a rodent, snoring humpback whale etc.

Auditory noise, such as e.g. auditory white noise may comprise, or sound similar to, the hissing spray of an aerosol can.

However, the noise may comprise perception stimuli emitted in form of light, i.e. visual noise such as background flicker or images which may comprise also moving images envisioned on a screen, or projected on a wall or similar by a projector means, comprised within, or attached to the noise generator 100. Further, visual noise may comprise light emitted in pulses in a stroboscopic manner, or alternatively slowly pulsating light in varying colours.

Further, according to some embodiments, the noise may comprise perception stimuli emitted in form of tactile stimulation, i.e. tactile noise. Such tactile noise may comprise vibrations, emitted through the air, through a chair the user 110 is sitting on, through the floor of the building the user 110 is visiting, or applied onto any body part of the user 110, according to different embodiments. According to some embodiments, tactile noise is transferred to the user 110 by means of a helmet, which helmet comprises vibrating elements configured to emit noise in form of vibrations, for example white noise vibrations of amplitude considered appropriate for the individual user 110. It is to be understood that when referring to a helmet herein, any other appropriate means for attaching tactile elements, such as e.g. a headband, a cap, a hat or similar. Actually, tactile elements may be attached directly to the skull of the user 110, e.g. by means of glue, adhesive tape, or other appropriate means of attachment, according to some embodiments.

In some embodiments, the noise may comprise perception stimuli emitted in form of vestibular noise. The vestibular nerves are known to influence neuronal circuits in the medullary cardiovascular areas and, through the cerebellar vermis, the basal ganglia and the limbic system. By means of vestibular noise, it may be possible to ameliorate the cognitive function of the user 110. According to some embodiments, vestibular noise may be emitted to the user 110 by electrical stimulation directly onto, or into, the user's head, i.e. skull.

In some further alternative embodiments, the noise may comprise perception stimuli emitted in form of haptic noise. The haptic noise may comprise vibrations emitted through the buttons of a keyboard, used by the user 110 for inputting data to a computer, phone or similar devise. However, the haptic noise may be emitted to the user 110 in several other manners.

It is further to be noted that the perception stimuli, or noise, to be emitted from the noise generator 100 may comprise one singular noise type, or kind of noise enumerated above. However, in some embodiments a combination of some, or all of the above enumerated kinds of noise may be emitted by the noise generator 100.

Independent of the selection of noise type as enumerated above, the noise may be emitted in form of e.g. white noise, according to some embodiments.

White noise is a random signal with a flat power spectral density. Thus, the signal may comprise equal power within a fixed bandwidth at any centre frequency. White noise draws its name from white light in which the power spectral density of the light is distributed over the visible band in such a way that the eye's three colour receptors (cones) are approximately equally stimulated. A random signal may be considered as white noise if it is observed to have a flat spectrum over a medium's widest possible bandwidth. Gaussian white noise may be seen as a particular form of white noise. White noise may be generated by combining sounds of all different frequencies together. If all of the imaginable tones that a human can hear where replayed at appropriate amplitude and independent from each other, white noise, or a sound effectively similar to white noise would be the result.

The adjective “white” is in this context utilized to describe this type of noise because of the way white light works. White light is light that is made up of all of the different colours, i.e. frequencies of light combined together. A prism or a rainbow separates the white light back into its component colours. In the same way, auditory white noise is a combination of all of the different frequencies of sound. White noise may be generated by playing e.g. 20,000 tones all playing at the same time.

According to some embodiments, the noise may be emitted in form of pink noise. Pink noise is a signal or perception stimuli with a frequency spectrum such that the power spectral density is inversely proportional to the frequency. In pink noise, each octave carries an equal amount of noise power. The name arises from being intermediate between white noise (1/f⁰) and red noise (1/f²) that is commonly known as Brownian noise, or brown noise.

Some embodiments of the invention may be based on the emission of brown noise. Brown noise is the kind of signal noise produced by Brownian motion, hence its alternative name of random walk noise. Brown noise may also be referred to as red noise.

Further according to some embodiments, the noise may be emitted in form of Johnson-Nyquist noise. Johnson-Nyquist noise is the electronic noise generated by the thermal agitation of the charge carriers such as e.g. electrons inside an electrical conductor at equilibrium, which happens regardless of any applied voltage. The generic, statistical physical derivation of this noise may be referred to as the Fluctuation-dissipation theorem, where generalized impedance or generalized susceptibility may be used to characterize the medium.

Also, the emitted noise may comprise thermal noise. Thermal noise in an idealistic resistor is approximately white, meaning that the power spectral density is nearly constant throughout the frequency spectrum. Additionally, the amplitude of the signal has very nearly a Gaussian probability density function, thereby resembling of white Gaussian noise.

The noise may furthermore be of shot noise type. Shot noise is a type of electronic noise that may be dominant when the finite number of particles that carry energy, such as e.g. electrons in an electronic circuit or photons in an optical device, is sufficiently small so that uncertainties due to the Poisson distribution, which describes the occurrence of independent random events, are of significance. The term shot noise may also be utilized to describe any noise source, even if solely mathematical, of similar origin.

Yet it is time to get further introduced to the actions comprised in the method according to embodiments of the invention.

FIG. 2 presents a method in a noise generator 100 for enhancing a cognitive function of a user 110. The cognitive function may comprise, but is not limited to memory, attention, perception etc.

In order to successfully enhance the cognitive function of the user 110, the method may comprise a number of actions 201-205, to be performed. However, it is to be noticed that they according to some embodiments may be performed in a somewhat different order than the enumerated order. Some of the actions 201-205 may be performed simultaneously, for example, or in a rearranged chronological order. Also, it is further to be noticed that some actions such as e.g. action 205 may only be performed within some embodiments. The method may comprise the following actions:

### Action 201

An attention level of the user 110 is estimated.

The attention level estimation may be continuously performed, enabling emission of continuously updated noise amplitude to be received by the user 110.

The attention level of the user 110 may be estimated based the user's brain activity, measured e.g. by any of: Electroencephalography, EEG, Magnetoencephalography, MEG, Magnetic Resonance Imaging, MRI, functional Magnetic Resonance Imaging, fMRI, Positron Emission Tomography, PET, X-ray Computed Tomography, CT, infrared measurement according to different embodiments.

However, the attention level of the user 110 may be estimated based on any of a cognitive test or a self-assessment test made by the user 110. According to some embodiments, the estimation of the attention level may be continuously evaluated according to some embodiments, e.g. by a computer program.

### Action 202

An optimal cognitive performance level of the user 110 is determined, without noise.

The optimal cognitive performance level of the user 110 to be determined may comprise the highest cognitive performance level of the user 110, such as e.g. the maximum cognitive performance level obtainable for the particular user 110.

### Action 203

A noise amount and a noise amplitude that is associated with the optimal cognitive performance level of the user 110 is predicted. This prediction is based on the estimated 201 attention level of the user 110.

The amount of noise is the length in time of the white noise to be applied.

The amplitude is the magnitude of change in the oscillating variable with each oscillation within an oscillating system. For example, when emitting auditory white noise, i.e. sound waves in air are oscillations in atmospheric pressure and their amplitudes are proportional to the change in pressure during one oscillation.

The prediction of noise amount and noise amplitude may be continuously performed, enabling emission of continuously updated noise amount and noise amplitude to be received by the user 110, according to some embodiments.

The determined noise amount and noise amplitude may render the highest cognitive performance level of the user 110 according to some embodiments, i.e. the optimal cognitive performance level of the user 110.

The action of predicting the noise amount and noise amplitude may further comprise determining a noise type, such as e.g. auditory noise, visual noise, haptic noise etc., according to some embodiments.

By estimating the attention level of the user 110, and also determining the optimal cognitive performance level of the user 110, it is possible to select an appropriate noise, particularly adapted for that individual user 110. Note that in some cases, i.e. when the user 110 already has the highest attention, the conclusion may be that no noise at all is to be emitted. For other users 110, based on the estimated attention level, the appropriate amount of noise and e.g. type of noise, and also the amplitude of the noise is determined.

### Action 204

Noise of the predicted 203 noise amount and noise amplitude is emitted, to be received by the user 110.

The noise to be emitted may comprise any, some or all of: white noise, pink noise, brown noise, red noise, flicker noise, random walk noise, Johnson-Nyquist noise, thermal noise, Shot noise, music, and/or sound of rain.

Further, the noise to be emitted may comprise any, some or all of: auditory noise, visual noise, tactile noise, vestibular noise, haptic noise, and/or subliminal noise, according to different embodiments.

The noise may be emitted according to some embodiments, to be received by the user 110 through any of vestibular stimulation to the brain of the user 110, or Transcranial Magnetic Stimulation, TMS.

### Action 205

This action is optional and may be performed only within some embodiments.

The noise generator 100 may discontinue emitting 204 noise when either the estimated 201 attention level of the user 110 exceeds a threshold value, or the cognitive performance level of the user 110 has reached the optimal cognitive performance level, according to some embodiments.

FIG. 3A is a block diagram illustrating an embodiment of a noise generator 100, and an arrangement 300 within the noise generator 100.

The arrangement 300 is configured to perform any, some or all of the actions 201-205 for enhancing a cognitive function of a user 110.

For the sake of clarity, any internal electronics or other components of the noise generator 100, not completely indispensable for understanding the present method has been omitted from FIG. 3A.

In order to perform the actions 201-205 correctly, the arrangement 300 comprises a processing circuit 320, configured to estimate an attention level of the user 110. Further, the processing circuit 320 is also configured to determine an optimal cognitive performance level of the user 110. In addition, the processing circuit 320 is further configured to predict a noise amount and a noise amplitude that is associated with the optimal cognitive performance level of the user 110, based on the estimated attention level of the user 110.

The processing circuit 320, may optionally further be configured to estimate the attention level of the user 110, based on the user's brain activity, according to some embodiments. Further, the processing circuit 320, may in addition be configured to discontinue emitting noise when either the estimated attention level of the user 110 exceeds a threshold value, or the cognitive performance level of the user 110 has reached the optimal cognitive performance level.

The processing circuit 320 may comprise e.g. one or more instances of a Central Processing Unit (CPU), a processing unit, a processor, a microprocessor, or other processing logic that may interpret and execute instructions. The processing circuit 320 may further perform data processing functions for inputting, outputting, and processing of data comprising data buffering and device control functions, such as call processing control, user interface control, or the like.

Also, the arrangement 300 comprises a noise emitting unit 330, configured to emit noise at the predicted noise amount and noise amplitude level, to be received by the user 110.

The noise emitting unit 330, may further be configured to emit noise of a type, comprising any, some or all of: white noise, pink noise, brown noise, red noise, flicker noise, random walk noise, Johnson-Nyquist noise, thermal noise, Shot noise, music, sound of rain.

The noise emitting unit 330, may further be configured to emit noise of a type, comprising any, some or all of: auditory noise, visual noise, tactile noise, vestibular noise, haptic noise, and/or subliminal noise. Thereby, the noise emitting unit 330, may emit noise of different amplitude, and amount.

Embodiments of the noise emitting unit 330, may further be configured to emit noise to be received by the user 110 through any of (electric, stochastic current) vestibular stimulation to the brain of the user 110, or Transcranial Magnetic Stimulation, TMS.

The arrangement 300 may further comprise a reception unit 310, configured to receive measurements related to the user's brain activity, according to some embodiments. Such measurements may be based on any of: Electroencephalography, EEG, Magnetoencephalography, MEG, Magnetic Resonance Imaging, MRI, functional Magnetic Resonance Imaging, fMRI, Positron Emission Tomography, PET, X-ray Computed Tomography, CT, infrared measurement.

According to some embodiments, the arrangement 300 may comprise at least one memory 325. The memory 325 may comprise a physical device utilized to store data or programs i.e. sequences of instructions, on a temporary or permanent basis. According to some embodiments, the memory 325 may comprise integrated circuits comprising silicon-based transistors. Further, the memory 325 may be volatile or non-volatile. The noise generator 100 may further according to some embodiments comprise e.g. one volatile memory 325 and also at least one non-volatile memory 325, to mention an example of an arbitrary set-up.

It is to be mentioned that any of the reception unit 310, the processing circuit 320, the memory 325, the noise emitting unit 330 and/or the noise generator 100 may be comprised in a helmet 120, to be worn by the user 110, according to some alternative embodiments, which are further presented and illustrated in FIG. 3C.

Embodiments of the noise generator 100 may comprise, or be comprised within any of: a computer, a media player, a mobile telephone, a gaming device, a helmet 120, a cap, a fan comprising a speed switch, a television set, a stroboscope and/or a noise enhancing means.

Further, it is to be noted that some of the described units 310-330 comprised within the arrangement 300 in the noise generator 100 are to be regarded as separate logical entities but not with necessity separate physical entities. To mention just one example, the reception unit 310 and the noise emitting unit 330 may be comprised or co-arranged within the same physical unit, a transceiver unit, which may comprise a transmitter circuit and a receiver circuit, which transmits outgoing radio frequency signals and receives incoming radio frequency signals, respectively, via an antenna according to alternative embodiments.

FIG. 3B illustrated an embodiment of a noise generator 100 comprising an arrangement 300, configured for performing at least some of the actions 201-205.

The noise generator 100 may comprise a portable electronic device, such as a media player, mobile telephone, a touch screen computer, or similar physical device.

The actions 201-205 to be performed in the noise generator arrangement 300 may be implemented through one or more processing circuits 320 in the noise generator 100, together with computer program code for performing the functions of the described actions 201-205. Thus a computer program, comprising instructions for performing the actions 201-205 in the noise generator 100 may perform the method for enhancing a cognitive function of a user 110, when being loaded into the one or more processing circuits 320 of the noise generator 100. Embodiments of the computer program may be referred to as an application, or app.

The computer program mentioned above may be provided for instance in the form of a data carrier carrying computer program code for performing at least some of the actions 201-205 according to some embodiments when being loaded into the processing circuit 320. The data carrier may be e.g. a hard disk, a CD ROM disc, a memory stick, an optical storage device, a magnetic storage device or any other appropriate medium such as a disk or tape that may hold machine readable data in a non-transitory manner. The computer program may furthermore be provided as computer program code on a server and downloaded to the noise generator 100 remotely, e.g. over an Internet or an intranet connection.

FIG. 3C illustrates yet an embodiment of a noise generator 100 comprising a helmet 120.

The helmet 120 may be worn by the user 110. An advantage with the helmet embodiment may be that different users in an environment like a class room or an office landscape may need different amplitude of noise. Thereby, if noise in form of e.g. auditory noise emitted by means of a loud speaker, it may be a problem to individualise the noise, adapted to each individual user 110.

According to some embodiments, the helmet 120 may comprise a hearing aid, e.g. a hearing aid configured to operate in conjunction with a hearing loop system, which hearing aid is configured for emitting white noise, and/or emitting white noise overlaid to the sound that is amplified. This may be made by fine tuning the signal to noise ratio of the hearing aid.

By using a helmet 120, feed-back information concerning each individual user's brain activity, such as e.g. the attention level of each user 110, may be acquired, and utilized for fine tuning the noise amplitude, considered appropriate for the individual user 110.

Thereby, also the emitted noise, adapted for the user 110, may be emitted through the helmet 120, to be received by the user 110. Thereby may it be possible to emit individually adapted noise to users 110 situated also in a public domain, without interfering with other individuals in the same area.

It is to be noted that the helmet 120 referred to herein may comprise any similar head worn gear, configured for emitting noise to the user 110 such as e.g. a head band, a hat, a cap, a hearing aid or electrodes fastened directly onto the skull of the user 110.

The terminology used in the detailed description of the particular exemplary embodiments illustrated in the accompanying drawings is not intended to be limiting of the methods and arrangements herein described.

As used herein, the singular forms “a”, “an” and “the” are intended to comprise the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. Furthermore, “connected” or “coupled” as used herein may include wirelessly connected or coupled. As used herein, the term “and/or” includes any and all combinations of one or more of the associated listed items.

1. A method in a noise generator, for enhancing a cognitive function of a user, the method comprising:
estimating attention level of the user,
determining an optimal cognitive performance level of the user in absence of noise,
predicting a noise amount and noise amplitude that is associated with the optimal cognitive performance level of the user, which prediction is based on the estimated attention level of the user,
emitting noise having the predicted noise amount and noise amplitude, to be received by the user.

2. The method according to claim 1, wherein the attention level estimation and the prediction of noise amount and noise amplitude are continuously performed, enabling emission of continuously updated noise amount and noise amplitude to be received by the user.

3. The method according to claim 1, wherein the attention level of the user is estimated based the user's brain activity, measured by any of: Electroencephalography, EEG, MEG, Magnetic Resonance Imaging, MRI, functional Magnetic Resonance Imaging, fMRI, Positron Emission Tomography, PET, X-ray Computed Tomography, CT, infrared measurement.

4. The method according to claim 1, wherein the attention level of the user is estimated based on any of a cognitive test or a self-assessment test made by the user.

5. The method according to claim 1, wherein the determined optimal noise amplitude and noise amount is rendering the highest cognitive performance level of the user.

6. The method according to claim 1, wherein the noise to be emitted comprises any, some or all of: white noise, pink noise, brown noise, red noise, flicker noise, random walk noise, Johnson-Nyquist noise, thermal noise, Shot noise, music, sound of rain.

7. The method according to claim 1, wherein the noise to be emitted comprises any, some or all of the following noise types: auditory noise, visual noise, tactile noise, vestibular noise, haptic noise, subliminal noise.

8. The method according to claim 1, wherein the noise is emitted to be received by the user through any of vestibular stimulation to the brain of the user, or Transcranial Magnetic Stimulation, TMS.

9. The method according to claim 1, wherein predicting noise amount and noise amplitude comprises selecting a noise type.

10. The method according to claim 1, further comprising discontinuing to emit noise when either the estimated attention level of the user exceeds a threshold value, or the cognitive performance level of the user has reached the optimal cognitive performance level.

11. An arrangement in a noise generator, for enhancing a cognitive function of a user, the arrangement comprising:
a processing circuit, configured to estimate an attention level of the user, and also configured to determine an optimal cognitive performance level of the user and to predict a noise amount and noise amplitude that is associated with the optimal cognitive performance level of the user, based on the estimated attention level of the user, and
a noise emitting unit configured to emit the predicted noise amount noise at the predicted noise amplitude level, to be received by the user.

12. The arrangement according to claim 11, wherein:
the processing circuit, is further configured to estimate the attention level of the user, based on the user's brain activity, and wherein the arrangement further comprises a reception unit, configured to receive measurements related to the user's brain activity, based on any of: Electroencephalography, EEG, Magnetoencephalography, MEG, Magnetic Resonance Imaging, MRI, functional Magnetic Resonance Imaging, fMRI, Positron Emission Tomography, PET, X-ray Computed Tomography, CT, infrared measurement.

13. The arrangement according to claim 11, wherein the noise emitting unit, is further configured to emit noise comprising any, some or all of: white noise, pink noise, brown noise, red noise, flicker noise, random walk noise, Johnson-Nyquist noise, thermal noise, Shot noise, music, sound of rain.

14. The arrangement according to claim 11, wherein the noise emitting unit, is further configured to emit noise comprising any, some or all of the following noise types: auditive noise, visual noise, tactile noise, vestibular noise, haptic noise, subliminal noise.

15. The arrangement according to claim 11, wherein:
the noise emitting unit, is further configured to emit noise to be received by the user through any of vestibular stimulation to the brain of the user, or Transcranial Magnetic Stimulation, TMS.

16. The arrangement according to claim 11, wherein the processing circuit, is further configured to discontinue emitting noise when either the estimated attention level of the user exceeds a threshold value, or the cognitive performance level of the user has reached the optimal cognitive performance level.

17. The arrangement according claim 11, wherein any of the reception unit, the processing circuit, the noise emitting unit and/or the noise generator is comprised in a helmet, to be worn by the user.

18. The arrangement according to claim 11, wherein the noise generator comprises any of: a computer, a media player, a mobile telephone, a gaming device, a helmet, a fan comprising a speed switch, a television set, a stroboscope.

19. A computer program for enhancing a cognitive function of a user, which computer program is configured to perform the method according to claim 1 when being loaded into a processing circuit.
